(19) United States
(12) Patent Application Publication
(10) Pub. No.: US 2012/0330162 A1
(43) Pub. Date: Dec. 27, 2012

(54) MODULATED APERTURE IMAGING FOR AUTOMATIC MOVING TARGET DETECTION

(75) Inventors: Nandini Rajan, Lincoln, MA (US); Sumanth Kaushik, Belmont, MA (US); Daniel Schuette, Arlington, MA (US)

(73) Assignee: Massachusetts Institute of Technology, Cambridge, MA (US)

(21) Appl. No.: 13/533,254

(22) Filed: Jun. 26, 2012

Related U.S. Application Data
(60) Provisional application No. 61/501,406, filed on Jun. 27, 2011.

(51) Int. Cl. A61B 6/00; H04N 7/18; G06K 9/00

(52) U.S. Cl. 600/476; 382/128; 348/78; 348/E07.085

(57) ABSTRACT

Traditional methods of detecting a moving target involve acquisition of video rate imagery in which data is acquired, stored, transmitted and then processed. Processing requires software for high precision frame-to-frame registration, detection and tracking. Example embodiments of the present invention include a method and an apparatus for generating instantaneous velocity maps that do not require acquisition, transmission, storing or processing of video-rate data. Incident radiation is directed onto one or more detectors, the detectors operating at a frame rate. The detectors acquire the first and second complementary sub-images of a single frame. The first and second complementary sub-images are combined to yield the change detection map. Example embodiments of the methods and devices described herein can be used in automatic detection of motion without tracking, optimization of image deblurring and optimization of detection of high speed and high frequency events, among others.

### RELATED APPLICATION

This application claims the benefit of U.S. Provisional Application No. 61/501,406, filed on Jun. 27, 2011. The entire teachings of the above application are incorporated herein by reference.

### BACKGROUND

Traditional methods for detecting the presence of moving targets have employed collecting full motion video at 30 frames per second (fps) or more followed by post processing. An alternate method employs Time Delay Integration (TDI), which is a method of increasing signal to noise ratio (SNR) at a cost of increased target blur. These methods typically require handling large amounts of data for storage, processing and transmission. The processing steps are computational intensive typically involving precise geo-registration, detection and tracking, followed by change detection.

### SUMMARY

An example embodiment of the present invention is a method and an apparatus for generating instantaneous velocity maps for both high speed events and for detecting resolved and unresolved objects that move by an amount corresponding to less than one resolvable spot of the detector during image acquisition.

One example embodiment of the present invention is a method of producing a change detection map. The method comprises directing incident radiation onto a detector, said detector having a frame rate; acquiring first and second complementary sub-images of a single frame, the first and the second complementary sub-images being acquired at a sub-frame rate; and combining the first and the second complementary sub-images to yield the change detection map.

Another example embodiment of the present invention is an apparatus for acquiring an image. The apparatus comprises a detector array configured to capture frames at a frame rate and to acquire first and second complementary sub-images, said detector array including a plurality of radiation exposure sites for converting the incident radiation into electric charges and a plurality of charge storage sites for storing electric charges, the detector array further configured to transfer, during acquisition of a single frame, the electric charges from the radiation exposure sites to the charge storage sites; and a processor, operably coupled to the detector array, configured to combine the first and the second complementary sub-images to yield the change detection map.

Another example embodiment of the present invention is an apparatus comprising a first and a second detector array, each having a frame rate; a first aperture configured to open for combined duration of one or more time intervals, said time intervals forming a first set of time intervals, thereby exposing the first detector to the incident radiation and capturing a first complementary sub-image; a second aperture configured to open for combined duration of one or more time intervals, said time intervals forming a second set of time intervals, thereby exposing the second detector to the incident radiation and capturing a second complementary sub-image; and a processor operably coupled to the first and second detector arrays and configured to combine the first and the second complementary sub-images to yield the change detection map.

Another example embodiment of the present invention is an apparatus comprising a detector array having a frame rate; a modulated polarizing element configured to (i) impose a first polarization onto the incident radiation for a combined duration of one or more time intervals, said time intervals forming a first set of time intervals, thereby forming a first beam having a first polarization and (ii) impose a second polarization onto the incident radiation for a combined duration of one or more time intervals, said time intervals forming a second set of time intervals, thereby forming a second beam having a second polarization; an optical element configured to direct the first beam and, separately, the second beam onto the detector, thereby acquiring the first and the second complementary sub-images; and a combiner, operably coupled to the detector array and configured to combine the first and the second complementary sub-images to yield the change detection map.

Another example embodiment of the present invention is an apparatus for producing a change detection map. The apparatus comprises means for directing incident radiation onto a detector, said detector having a frame rate; means for acquiring first and second complementary sub-images of a single frame, the first and the second sub-images being acquired at a sub-frame rate; and means for combining the first and second complementary sub-images to yield the change detection map.

Another example embodiment of the present invention is an apparatus for producing a change detection map. The apparatus comprises at least one detector array configured to acquire an image encoded in incident radiation, said detector array having a frame rate; a modulator, configured to divide the image encoded in the incident radiation into the first and the second complementary sub-images during a single frame acquisition period of the detector; and a combiner, operably coupled to the at least one detector array and configured to combine the first and the second complementary sub-images to yield the change detection map.

Another example embodiment of the present invention is a method of diagnosing a disorder in a subject. The method comprises detecting saccades of the subject by directing radiation reflected from at least one eye of the subject onto a detector, said detector having a frame rate; acquiring first and second complementary sub-images of a single frame, wherein the first and the second complementary sub-images are acquired at a sub-frame rate; and combining the first and the second complementary sub-images to detect the saccades of the subject, wherein the disorder is a traumatic brain injury, an attention deficit disorder, autism, dyslexia, multiple sclerosis or ocular palsy.

Another example embodiment of the present invention is a method of detecting saccades in a subject. The method comprises directing radiation reflected from at least one eye of the subject onto a detector, said detector having a frame rate; acquiring first and second complementary sub-images of a single frame, the first and the second complementary sub-images being acquired at a sub-frame rate; and combining the first and the second complementary sub-images to detect the saccades in the subject.

The example method and apparatus bypass both the storage requirements for full frame imagery data, and also the ground processing. Because the motion detection is accomplished on a per frame basis, the relative change in aspect angle of the sensor to the ground is minimal, and, therefore, co-registration is not required. Because the modulating sequence is generated to search for multi-target velocities, multi-hypothesis Kalman Filter tracking algorithms are not required. In addition, the output of the methods described herein includes automatic generation of instantaneous target velocities. Because full motion imagery is not required for the methods described herein, the resulting data is highly compressible compared to standard full frame video for transmission.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic diagram illustrating the use of a device of the present invention to detect a moving object.

FIG. 2 is a flow-chart illustrating an embodiment of a method of the present invention.

FIG. 3 is a schematic diagram depicting one embodiment of a device of the present invention.

FIG. 4A is a schematic diagram depicting one embodiment of a device of the present invention.

FIG. 4B is a schematic diagram depicting one embodiment of the detector array 401 shown in FIG. 4A.

FIG. 5 is a schematic diagram illustrating one embodiment of the detector array 401 shown in FIG. 4A.

FIG. 6A is a plot showing one embodiment of a dual-rail binary modulation pattern employed by an embodiment of the present invention.

FIG. 6B is a plot showing an alternative embodiment of a dual-rail binary modulation pattern employed by an embodiment of the present invention.

FIG. 6C is a plot of yet another alternative embodiment of a dual-rail binary modulation pattern employed by an embodiment of the present invention.

FIG. 7 is a screen capture of an output of MATLAB simulation of several Gaussian-shaped targets undergoing a range of linear motions from multi-pixel to sub pixel during a single image frame acquisition.

FIG. 8 is a simulated change detection map produced by the method of the present invention using the simulation shown in FIG. 7.

FIG. 9 is a diagram that shows four panels wherein each panel is a plot of pixel intensity measured along the lines shown in FIG. 8 in the directions of the arrows.

FIG. 10A is a schematic diagram of one embodiment of a device described herein.

FIG. 10B is an illustration of formation of two sub-images on a detector during the operation of the device shown in FIG. 10A.

FIG. 11 is a plot showing a voltage profile applied to a tunable wave plate embodiment of a modulated polarizing element of employed by the device shown in FIG. 10A.

FIG. 12 is a plot showing amplitudes of eye movements as a function of time, where the eye movements of a human subject were detected by an example embodiment of the present invention.

### DETAILED DESCRIPTION OF PREFERRED EMBODIMENTS

A description of example embodiments of the invention follows. The teachings of all patents, published applications and references cited herein are incorporated by reference in their entirety.

As used herein, the term “frame” means “one of the many unique still images which compose the complete moving picture.”

As used herein, the term “frame rate” means “the frequency at which an imaging device reproduces consecutive frames.”

As used herein, the term “complementary sub-image” means one of a pair (or more) of images, with each sub-image acquired during a single frame capture time interval, wherein the single frame acquisition period is the inverse of the frame rate. As used herein, the phrase “sub-frame rate” refers the frequency of image acquisition (i.e. the inverse of the time of a single frame acquisition) that is greater than the frame rate of the detector.

The sub-frame rate is the inverse of the maximum time resolution of the detector and is greater than the frame rate of the detector. The two or more sub-frame rate images are acquired during a single frame acquisition time.

As used herein, the term “lens element” refers to one or more elements having optical power, such as lenses, that alone or in combination operate to modify an incident beam of radiation, e.g. light, by changing the curvature of the wavefront of the incident beam of light.

As used herein, the term “modulator” refers to any device that can be configured to divide an image encoded in the incident radiation into the first and the second complementary sub-images during a single frame acquisition period of the detector.

Examples of modulators include a combination of two or more modulated apertures, a modulated polarizing element (e.g., a tunable wave plate), a detector that comprises separable photoelectron collection (incident radiation exposure) sites and charge storage sites, or any other modulated optical element capable of controllably separating or dividing an image encoded in the incident radiation into two sub-images. Such separation can be accomplished before acquisition of an image by the detector by, for example, spatial shearing, polarization splitting, or spectral separation of an incident beam into two beams. Such separation can be accomplished by the detector itself, after acquisition of an image by the detector, by performing a sequence of photoelectron collecting operations and charge storage operations, as will be described in detail below.

One example embodiment of a modulator is a combination of a first aperture configured to open for a combined duration of one or more time intervals, said time interval(s) forming a first set of time intervals, thereby exposing the first detector to the incident radiation and capturing a first complementary sub-image, and a second aperture configured to open for a combined duration of one or more time intervals, said time interval(s) forming a second set of time intervals, thereby exposing the second detector to the incident radiation and capturing a second complementary sub-image.

In this example embodiment, either the lengths of time intervals in each set of time intervals can be modulated, or the size of the aperture can be modulated, or both. The values of the modulated properties in each set can be the same or different; these values can also be constant or random. Where the values are random, such values can be selected from any random variable distribution, e.g. from an exponential distribution. In one embodiment, the modulated values are modulated according to a dual-rail binary modulation pattern.

Another example embodiment of a modulator is a modulated polarizing element configured to (i) impose a first polarization onto the incident radiation for a combined duration of one or more time intervals, said time intervals forming a first set of time intervals, thereby forming a first beam having a first polarization and (ii) impose a second polarization onto the incident radiation for a combined duration of one or more time intervals, said time intervals forming a second set of time intervals, thereby forming a second beam having a second polarization. Separate detection of the first and the second beams results in acquisition of the first and the second sub-images, respectively.

In this example embodiment, the lengths of time intervals in each set of time intervals can be modulated. The values of the modulated properties in each set can be the same or different; these values can also be constant or random. Where the values are random, such values can be selected from any random variable distribution, e.g. from an exponential distribution. In one embodiment, the modulated values are modulated according to a dual-rail binary modulation pattern.

Yet another example embodiment of a modulator is a detector array that includes a plurality of radiation exposure sites for converting the incident radiation into electric charges (i.e., for collecting photoelectrons) and a plurality of charge storage sites for storing electric charges, the detector array further configured to transfer, during acquisition of a single frame, the electric charges from the radiation exposure sites to the charge storage sites.

In this example embodiment, the lengths of time intervals during which photoelectron are collected at the radiation exposure sites before being transferred to the charge storage sites can be modulated. The values of the modulated properties can be the same or different; these values can also be constant or random. Where the values are random, such values can be selected from any random variable distribution, e.g. from an exponential distribution. In one embodiment, the modulated values are modulated according to a dual-rail binary modulation pattern.

As used herein “a modulated polarizing element” refers to a device that can controllably impart polarization onto incident radiation. An example of a modulated polarizing element is a tunable wave plate.

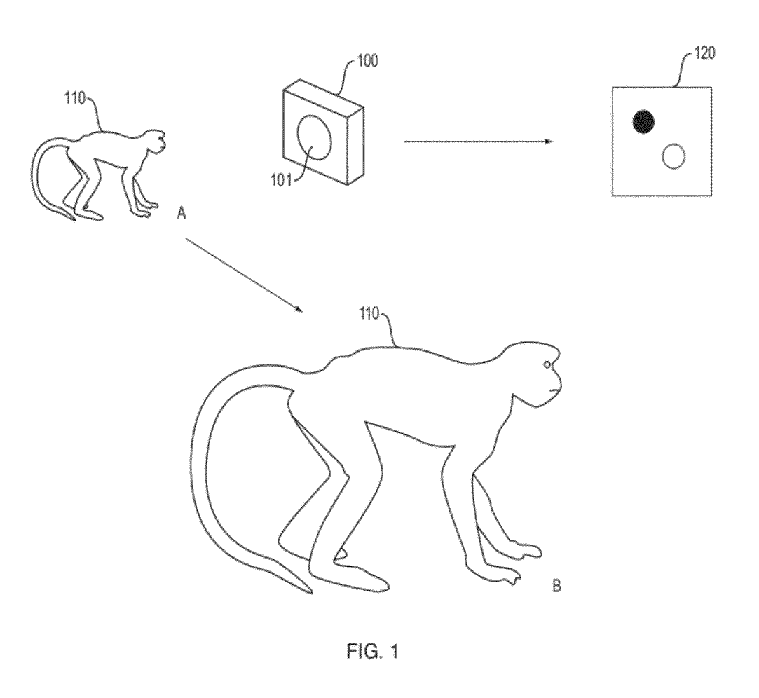

An embodiment of the present invention is a method and an apparatus for obtaining an instantaneous object velocity map, also referred to herein as a “change detection map.” Referring to FIG. 1, a device 100 is positioned so that its objective 101 can capture the image of an object 110. When the object 110 moves from position A to position B, device 100 outputs an instantaneous velocity map 120 that shows the direction of movement of object 110 as well as its speed.

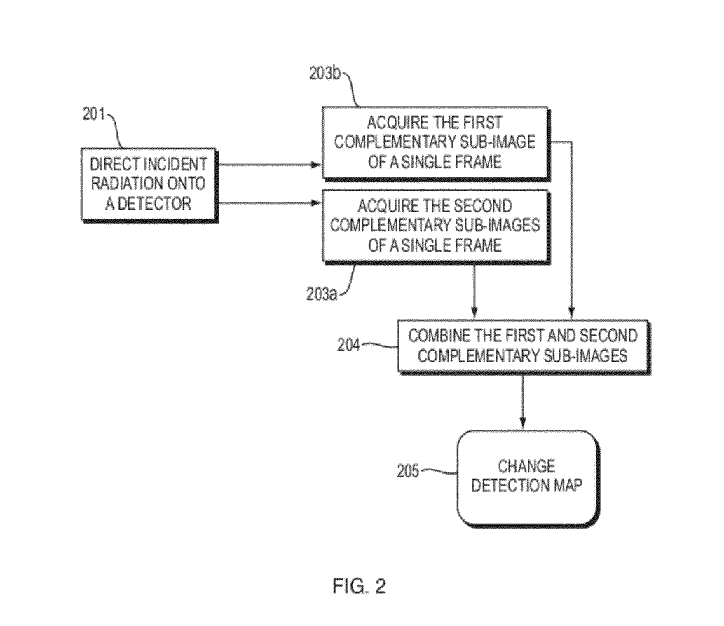

In one embodiment, the present invention is a method of producing a change detection map. Referring to FIG. 2, the method directs incident radiation onto one or more detectors to capture a frame (201). The one or more detectors, which can be charge-coupled devices, operate at a specified frame rate, e.g., 30 frames per second (fps). The one or more detectors acquire the first and second complementary sub-images of a single frame (203A and 203B) at a sub-frame rate. The first and second complementary sub-images are combined (204) to yield the change detection map 205.

In various embodiments, the present invention is a method of producing a change detection map. During the operation of the method, incident radiation is directed onto one or more detectors to capture a frame. The detector has a frame rate. First and second complementary sub-images of a single frame are acquired by the one or more detectors, wherein the first and the second sub-images are acquired at a sub-frame rate. The first and the second complementary sub-images are combined to yield the change detection map.

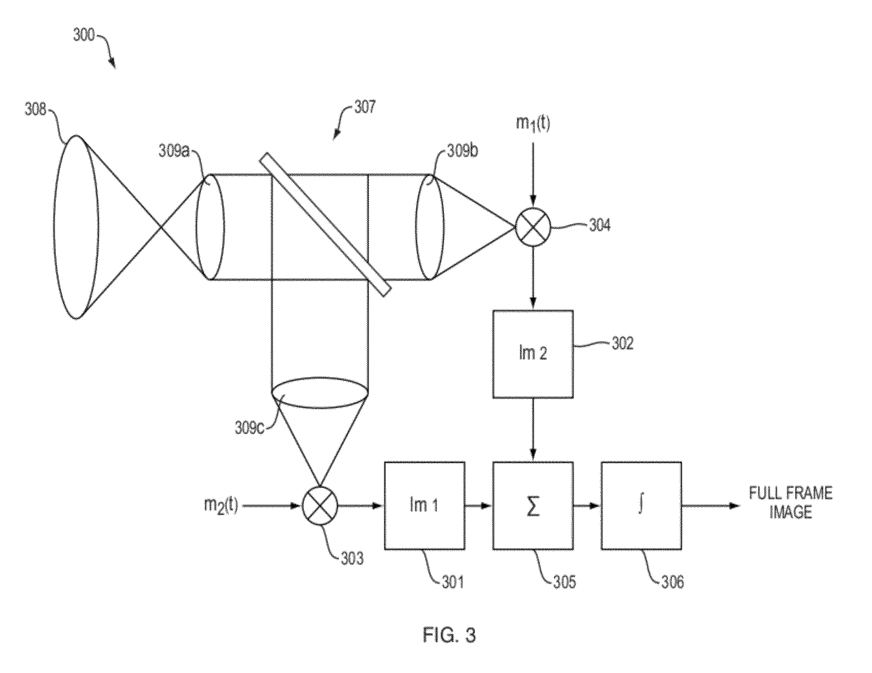

In example embodiments, the methods of the present invention employ a device that includes two or more apertures. Certain examples of such devices and their methods of operation will be explained in greater details below, for example with reference to FIG. 3. Generally, in such embodiments, acquiring the first and the second complementary sub-images includes directing the incident radiation through two or more apertures. The first aperture can be opened for a combined duration of one or more time intervals, said time intervals forming a first set of time intervals. The second aperture can be opened for a combined duration of one or more time intervals, said time intervals forming a second set of time intervals. The lengths of time intervals in each set of time intervals are adjustable. In one example embodiment, the lengths of the intervals between opening and closing of the shutters are chosen to be constant. This embodiment is suitable for objects moving in cyclical periodic manner. In another example embodiment, the lengths of the intervals are chosen randomly from a statistical distribution. This embodiment is more appropriate for targets moving in unknown manner and direction.

In example embodiments of the above-described methods employing a device that includes at least two apertures, the first aperture includes a first shutter and the second aperture includes a second shutter, wherein opening the first and the second apertures includes actuating the first and the second shutters.

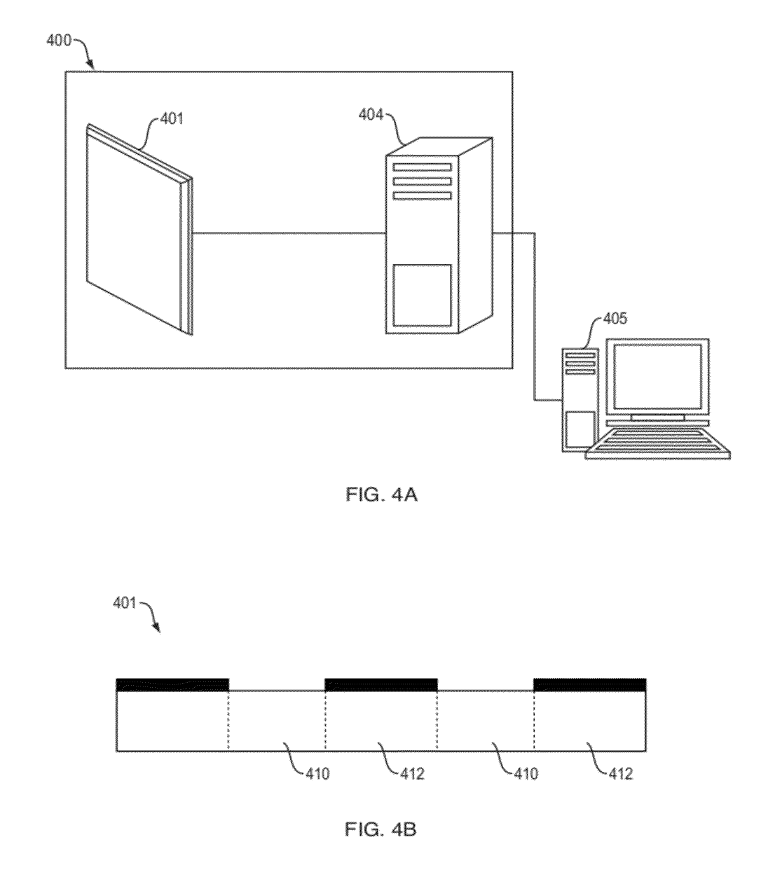

In alternative example embodiments, the methods of the present invention employ devices that are configured to convert the incident radiation into electric charges and to store the electric charges. Certain examples of such devices and their methods of operation will be explained in greater details below, for example with reference to FIG. 4A and FIG. 4B. Generally, in such embodiments, acquiring the first and the second complementary sub-images includes, during the single exposure: converting the incident radiation into first set of electric charges representing the first sub-image; storing the first set of charges; and converting the incident radiation into second set of electric charges representing the second sub-image. Acquiring the first and the second complementary sub-images can include directing the incident radiation at a detector array that includes a plurality of radiation exposure sites for converting the incident radiation into electric charges and a plurality of charge storage sites for storing electric charges. Storing the first set of electric charges can include transferring the first set of electric charges from the radiation exposure sites to the charge storage sites. Converting the incident radiation into the first set of electric charges includes exposing the exposure sites to the incident radiation for combined duration of one or more time intervals, said time intervals forming a first set of time intervals; converting the incident radiation into the second set of electric charges includes exposing the exposure sites to the incident radiation for combined duration of one or more time intervals, said time intervals forming a second set of time intervals. The lengths of time intervals in each set of time intervals are adjustable. For example, the lengths of time intervals in each set are modulated according to a dual-rail binary modulation pattern. In one example embodiment, the lengths of time intervals are determined by a discrete uniform distribution drawn from the sample {−1, 0, 1} or a Rademacher distribution drawn from the sample {−1, 1}.

In various embodiments of the present methods, combining the first and second complementary sub-images includes adding the first complementary sub-image from the second complementary sub-image. In other embodiments, combining the first and second complementary sub-images includes subtracting the first complementary sub-image from the second complementary sub-image.

In certain embodiments, combining the first and second complementary sub-images to yield the change detection map includes adding the first and second complementary sub-images to yield a complementary sub-image sum and integrating the complementary sub-image sum to yield the change detection map. In further embodiments, the methods of the present invention include estimating motion of an object using the change detection map. In example embodiments, the object being detected moves by an amount corresponding to less than one resolvable spot of the detector.

An embodiment of the present invention is a device 300 shown in FIG. 3. The device 300 comprises the first detector array 301 and the second detector array 302, each having a frame rate. The device 300 further includes the first aperture 303, controlled, e.g., by a shutter, configured to modulate radiation incident on the first detector array 301 at a sub-frame rate to produce a first complementary sub-image, and the second aperture 304, controlled, e.g., by a shutter, configured to modulate radiation incident on the second detector array 302 at the sub-frame rate to produce a second complementary sub-image. The device 300 further includes a combiner 305 operably coupled to the first detector array 301 and the second detector array 302 and configured to combine the first and the second complementary sub-images. The device 300 can further include an integrator 306 operably coupled to a combiner 305 and configured to integrate an output of a combiner 305 to form a change detection map. In certain specific embodiments, the device 300 can further include a processor operably coupled to the combiner 305 and configured to estimate motion of an object using the change detection map. The processor can be configured to estimate motion of an object. The object can move by an amount corresponding to less than one resolvable spot of the detector array during acquisition of a single frame. The embodiment of the device 300 shown in FIG. 3 includes a beam splitter 307, an objective 308, and one or more lens elements 309 (lens elements 309a, 309b and 309c are shown).

During the operation of device 300, apertures 303 and 304 are each modulated on a “per frame” basis. During a single frame acquisition time, the aperture 303 and 304 is open or closed for the duration of time that is less than the single frame acquisition rate. For each image frame, the input signal is modulated by two separate time sequences using two separate optical modulators such as those shown as apertures 303 and 304. The optical modulators modulate the amplitude of the light impinging on the detector arrays in either discrete (on or off) steps, or over a continuous range, e.g., (between 0 and 1, where 0 represents a state in which no photons impinge upon the detector array and 1 represents a state in which all photons pass through the optical modulator(s) on a path to the corresponding detector array(s)). The two images captured by detector arrays 301 and 302 are then added by the combiner 305 and the data from the full frame is integrated by the integrator 306 to generate a single change detection image.

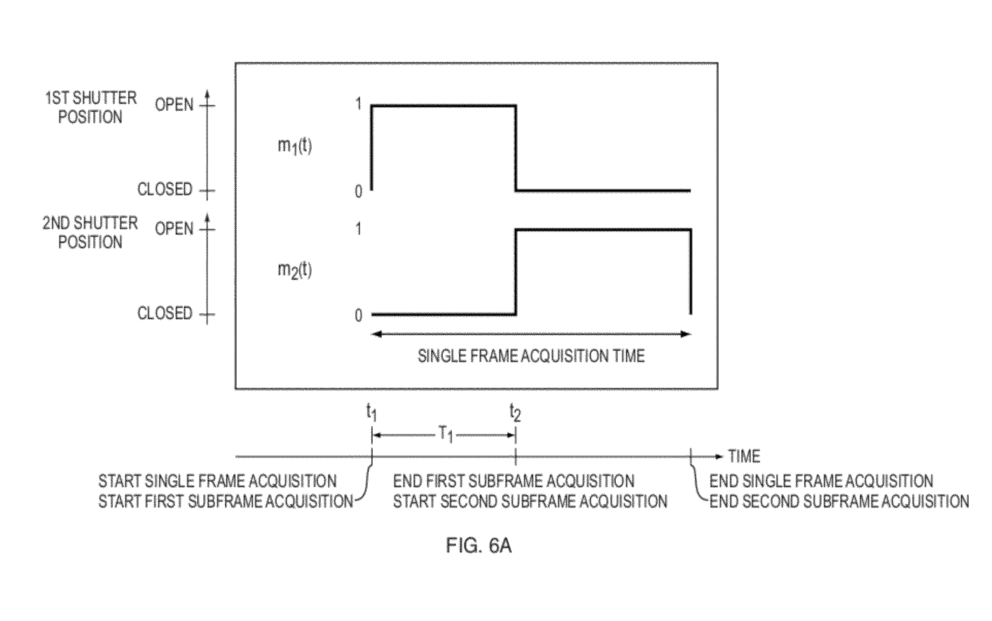

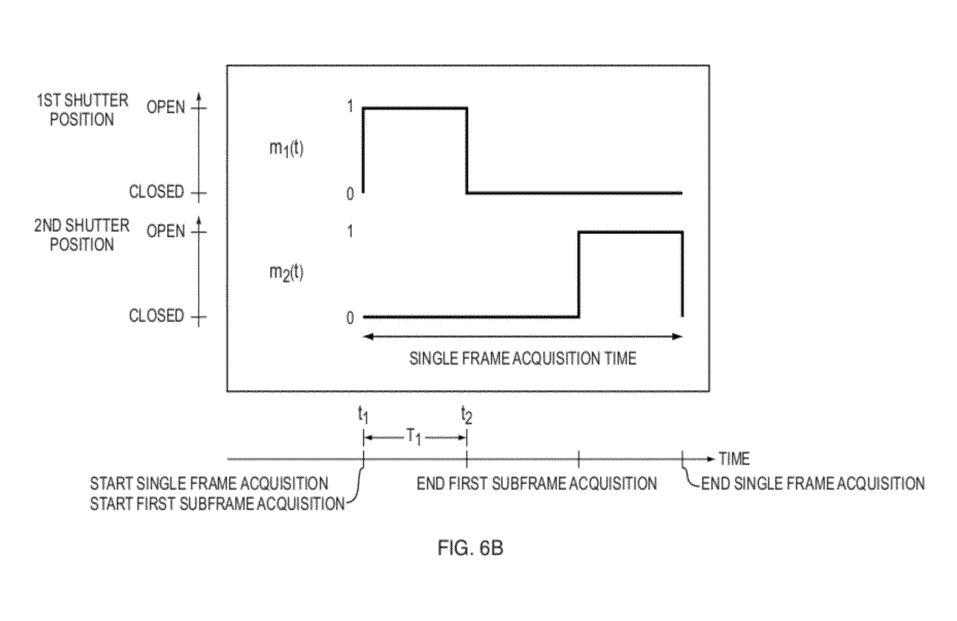

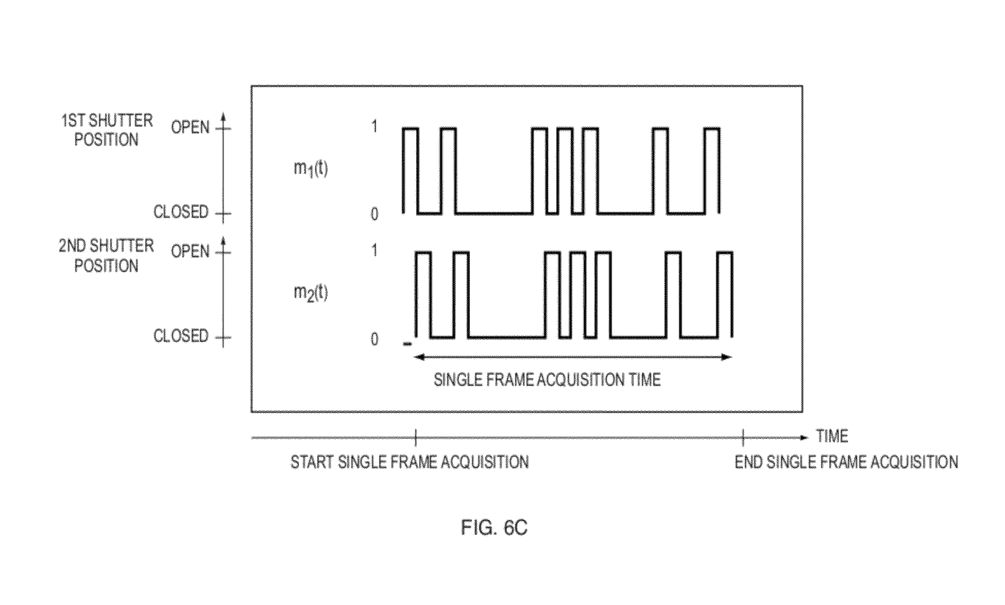

In a specific embodiment, at least one aperture 303 or 304 is opened and closed according to a dual-rail binary modulation pattern. For example, the dual-rail binary modulation pattern of the apertures 303 and 304 are given by dividing the acquisition time of a single frame into two intervals. Referring to FIG. 6A, modulation sequence m₁(t) describes the “open” and “closed” positions of the first aperture (“1” and “0”, respectively). For m₁(t), the first time interval corresponds to +1 value, the second time interval corresponds to 0. Modulation sequence m₂(t) describes the “open” and “closed” positions of the second aperture (“1” and “0”, respectively). For the modulation sequence m₂(t), the first time interval corresponds to 0 and the second time interval corresponds to +1. As a result, the sequences m₁(t) and m₂(t) are complementary.

An alternative embodiment of the dual-rail binary modulation pattern is shown in FIG. 6B. Here, the acquisition time of a single frame is divided into three intervals. The duration of the first and last intervals is the same. The duration of the second time interval can be variable. In FIG. 6B, as in FIG. 6A, the modulation sequence m₁(t) describes the “open” and “closed” positions of the first aperture (“1” and “0”, respectively), and m₂(t) describes the “open” and “closed” positions of the second aperture (“1” and “0”, respectively). For modulation sequence m₁(t), the first time interval corresponds to +1 and the second and third time intervals correspond to 0. For the modulation sequence m₂(t), the first two time intervals correspond are set to 0 and the third time interval corresponds to 1. In yet another example embodiment, the lengths of time intervals during which the apertures stay open are given by a random variable distribution. In other words, the T₁ is a random variable.

For example, the duration of time intervals during which the apertures are open can be based on a discrete uniform distribution over a sample {−1, 0, 1} or Rademacher distribution over a sample {−1, 1}. If T is the single frame acquisition time and Tₘᵢₙ is the minimum time resolution for the shuttering of the apertures, the number of chops N can be defined as T/Tₘᵢₙ. The sequence of aperture openings can be parameterized as follows. Let a sequence m(n), where n is the index from 1 to N, where N is defined as the number of chops, be drawn from a discrete random distribution over the sample {−1, 0, 1} or a Rademacher distribution over {−1, 1}, such that the sum of m(n) is equal to 0. Then the modulation sequence m₁(t), where t is a continuous time variable between Tₘ*(n−1) and Tₘᵢₙ*n, can then be set to 0 if m(n) is 0 or −1 and 1 if m(n) is 1. The modulation sequence m₂(t) can be set to 0 if m(n) is 0 or 1, and to 1 if m(n) is −1. Consequently, when each modulation sequence m₁(t) and m₂(t) is integrated over the frame acquisition time, and the two values of the resulting integrals are subtracted, the result of this subtraction equates to zero.

FIG. 6A illustrates an example embodiment of the above-described procedure. The sample sequence m(n) that resulted in generating the two modulation sequences m₁(t) and m₂(t) was drawn from the discrete random distribution from sample {−1, 0, 1}.

In the procedure described above, the number of chops N is a natural number greater than two. Selection of the natural number N is well within the skill of a person of ordinary skill and the number N can be adjusted based on the properties of the image acquisition devices, the speed of the events being detected, the lighting conditions, etc.

One of ordinary skill in the art would be able to determine the sequence m(n) without undue experimentation, based on factors such as the properties of the image acquisition devices, the speed of the events being detected, the lighting conditions, etc. An advantage of the drawing the modulation sequence from the above-described discrete distributions is a large spread in sampling that permits the capture of multi-velocity targets. The described modulation patterns allow for motion capture of both high speed targets as well as sub-pixel movement.

Various embodiments of the present invention employ an apparatus 400 shown in FIG. 4A. The apparatus 400 comprises a detector array 401 configured to capture frames at a frame rate.

The detector array 401 is configured to convert the incident radiation into first set of electric charges representing first sub-image; to store the electric charges; and to convert the incident radiation into second set of electric charges representing the second sub-image. In example embodiment shown in FIG. 4B, the detector array 401 includes a plurality of radiation exposure sites 410 for converting the incident radiation into electrical charges and a plurality of charge storage sites 412 for storing electrical charges. For example, the detector array 401 can include a stripped light shield, so that the exposed rows form radiation exposure sites 410, and the shielded rows form charge storage sites 412. The detector array 401 is configured to transfer electric charges from the radiation exposure sites to the charge storage sites.

Referring to FIG. 4A, the device 400 can further include a combiner 404 operably coupled to the detector array 401 and configured to combine the first and second complementary sub-images to produce a change detection map. A processor 405, operably coupled to the combiner 404, can be configured to estimate motion of an object using the change detection map.

In various embodiments of the devices 300 and 400, the devices can include a processor configured to estimate motion of an object, wherein the object is resolved and moves at least one pixel during the acquisition time. Referring, for example, to FIG. 4A, the combiner 404 extracts and then subtracts the two complementary sub-images to generate a change detection map. The output of the combiner 404 (i.e. the generated change detection map) becomes the input to the processor 405, which determines the estimated motion of the object. The velocity of resolved targets is determined by measuring the peak amplitude spacing of complementary detections and multiplying by the sub-frame rate.

In example embodiments, the processor can be configured to determine the presence of a moving object wherein the object moves by an amount corresponding to less than one resolvable spot of the detector during acquisition of a single frame. Referring, for example, to FIG. 4A, the combiner 404 extracts and then subtracts the two complementary sub-images to generate a change detection map. The existence of background across which the targets move permits detection of movement. For example, all natural scenes have structure in the background. As a target moves across the background, it occludes portions of it. Acquisition of sub-images during a single frame acquisition permits detecting the temporal variability of this occlusion during transit of the target. This temporal variability is a measurable effect at a resolution lower than that of the detector. The existence of the unresolved target moving at less than one resolvable spot is indicated in the change detection map.

The operation of an example embodiment of the devices of the present invention, such as the device 400 shown in FIG. 4A and FIG. 4B, will now be described. During the operation of the embodiment of the device 400 that employs the detector array shown in FIG. 4B, generation of the two sub-images occurs on the detector array by way of employing a plurality of radiation exposure sites 410 for converting the incident radiation into electric charges and a plurality of charge storage sites 412 for storing electric charges.

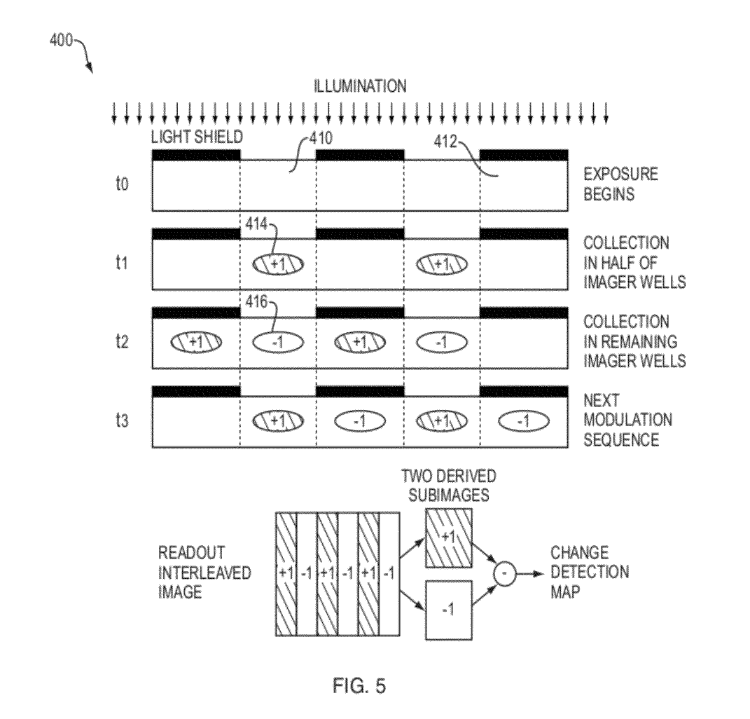

Referring to FIG. 5, the detector array 400 can include striped light shield, so that the exposed rows form a plurality of radiation exposure sites 410 and the shielded rows form a plurality of charge storage sites 412. (FIG. 4B and FIG. 5 each show a portion of the section of the detector array 400 that includes striped light shield, cut across the stripes.) At the initial exposure time, t₀, the plurality of radiation exposure sites 410 are illumination by incident radiation. This exposure generates charges 414, shown symbolically as “+1,” that represent the first sub-image. At time t₁, subsequent to t₀, charges 414 are shifted to charge storage sites 412. At time t₂, subsequent to t₁, the plurality of radiation exposure sites 410 are again illuminated, thus generating a second plurality of charges 416, symbolically shown as “−1,” representing the second sub-image. At time t₃, subsequent to t₂, charges 414 and 416 can be shifted again or be collected for processing into first and second sub-images. Shifting charges between subsets of the radiation exposure sites 410 and the charge storage sites 412 permits acquisition of more than two sub-images. Charges can be shifted at a sub-frame rate of the detector array. After the acquisition time of a single frame, the charges are all shifted out onto the read register. The resultant data is row-interleaved, having time histories of exposure times during the single frame acquisition time. The interleaved data can then be separated into the two sub-images by a combiner 404 that subtracts the two sub-images to generate a change detection map. A processor 405, operably connected to the combiner 404, can be utilized to generate an instantaneous velocity estimate.

The duration and sequence of sub-image acquisition by each subset of the plurality of the radiation exposure sites 410 as well as the sequence of charge transfer operations from the radiation exposure sites 410 to the charge storage sites 412 can be determined by a modulation pattern. When T is the single frame acquisition time and Tₘᵢₙ is the minimum time required for the transfer of charges from the radiation exposure sites to the charge storage sites, the single frame acquisition time T can be divided into N time intervals (“chops”), where the number N is defined to be T/Tₘᵢₙ. Then a modulation sequence m(n), where n is the index from 1 to N, is defined by drawing from a Rademacher distribution over the sample {−1, 1} such that the sum of all values of m(n) over all values of n is equal to 0. Each of the N chops is then assigned the number, “−1” or “+1” in order corresponding to the modulating sequence m(n). The first chop, for example, can be assigned number “+1.” Photoelectrons are collected at the first subset of the radiation exposure sites for the combined duration of the time intervals labeled “+1,” thereby acquiring the first sub-image. Similarly, photoelectrons are collected at the second subset of the radiation exposure sites for the combined duration of the time intervals labeled “−1,” thereby acquiring the second sub-image. The charges are shifted from each subset of radiation exposure sites and to the charge storage sites at the times that correspond to change of chop labels from “+1” to “−1” or from “−1” to “+1.”

In various embodiments of the present invention, whenever a shutter is employed to open or close an aperture, whether device 300 of FIG. 3 or in device 400 of FIG. 4A, the shutter can be selected from the group consisting of an electro-optic device, magneto-optic device, liquid crystal device, and a mechanical shutter. Other forms of shutters currently known in the art or later developed may also be employed, optionally in various combinations.

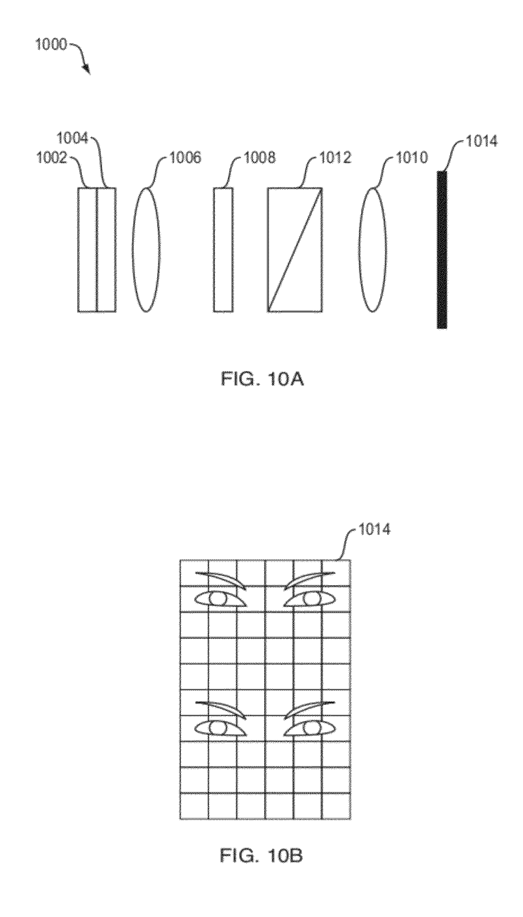

As described above, in one example embodiment, a device described herein employs as a modulator a modulated polarizing element configured to (i) impose a first polarization onto the incident radiation for combined duration of one or more time intervals, said time intervals forming a first set of time intervals, thereby forming a first beam having a first polarization and (ii) impose a second polarization onto the incident radiation for combined duration of one or more time intervals, said time intervals forming a second set of time intervals, thereby forming a second beam having a second polarization. An example of such a device is shown in FIG. 10A.

A device 1000 as shown in FIG. 10A comprises a polarizer 1004, an objective lens element 1006, a modulated polarizing element 1008 (e.g., an electrically tunable wave plate), a beam splitter 1012 and a detector 1014. The device 1000 can also include optional elements, such as filter 1002 (e.g. an infrared filter) and one or more lens elements 1010. Additionally an optional light source (e.g. a pulse infrared LED) can be included.

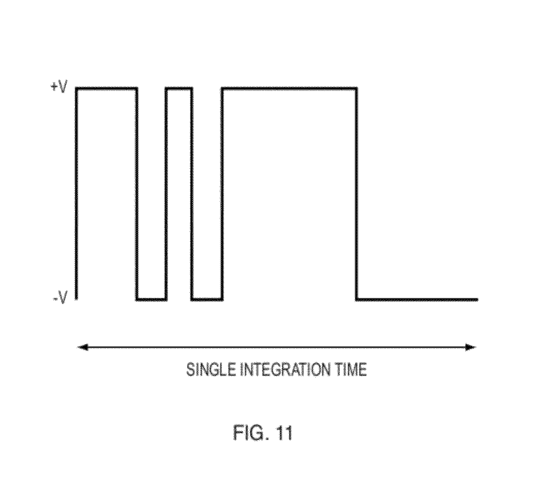

A modulated polarizing element 1008 is configured to impose a first polarization onto the incident radiation for combined duration of one or more time intervals, said time intervals forming a first set of time intervals, thereby forming a first beam having a first polarization; and to impose a second polarization onto the incident radiation for combined duration of one or more time intervals, said time intervals forming a second set of time intervals, thereby forming a second beam having a second polarization. For example, referring to FIG. 11, a voltage profile shown in FIG. 11 can be applied to an electrically tunable wave plate. When applied, voltage “+V” will result in forming the first beam having the first polarization, while voltage “0” will result in forming the second beam having the second polarization. Following beam formation, both beams are directed at the beam splitter 1012, which, depending on the polarization of the beam, directs the first and the second beams at different locations on the detector 1014. Thus, the first and the second complementary sub-images are formed on detector 1016, as shown in FIG. 10B.

The methods and devices described herein can automatically generate an instantaneous change detection/velocity map. The end product (i.e. the change detection/velocity map) was previously obtained from full motion imagery processing, requiring challenging methods of precise registration, detection, and tracking, followed by motion segmentation. The example methods and devices described herein can be used for generation of instantaneous velocity maps for both high speed events, as well as for sub-pixel movers. Because the encoding is done on a “per frame” basis, a multi-mode camera function can be achieved by defining the modulation sequence at each frame cycle. For example, it is possible to interleave between standard full frame imaging and change detection modulation to achieve automatic mover (i.e. the image of the moving object) removal from imagery data. It is also possible to automatically fuse data products by interleaving the modulation sequence to generate a change detection map during one frame with a full frame image at the next consecutive frame. Because a change detection or velocity estimate is made within a single frame via sub-frame sampling, the relative movement of objects as well as sensor is potentially subpixel, and thus computationally intensive frame-to-frame registration is not required. High speed events can be detected on each frame, and then a specific code can be generated at next interval for optimized target deblurring by using techniques known in the art.

Another benefit of certain embodiments of the method is that it is possible to increase the readout times for scanning systems by dividing the modulation into two (or more) parts, the first part performing the change detection and the second modulating the aperture. Certain embodiments of the methods disclosed herein automatically generate the velocity map and make it available for determining the direction of deblurring.

The disclosed methods can be applied in activity-based video compression. Because a mover (i.e. a moving target) location is determined, selective compression of the background without concomitant compression of the moving target is possible. Thus, the background can be coded at a lower resolution than the moving target, which can be coded with high fidelity.

The operation of a multi-resolution encoding procedure will now be described.

The output of the two modulation sequences m₁(t) and m₂(t) are two sub-images, S1 and SI. When the sub-images S1 and S2 are subtracted, a change detection map C, is generated. When the two sub-images are added, an image B, as acquired from a single frame acquisition time, is recovered. The change detection map C is sparse, i.e. it is primarily populated with zero-value elements, and hence is highly compressible.

Both change detection map C and the image B can be functions of time t. Change map C is a function of time: C=C(t) at time t; B=B(t) at time t. C(t) is defined as follows:

C(t)=sign(Abs(S1−S2)),

where, sign( ) is the signum function and Abs( ) is the absolute value function.

The change detection image C′ is also a function of time t: C′=C′(t). C′(t) is given by the following formula:

C′(t)=f(C(t)*B(t)),

where function f( ) is a linear or nonlinear map. In one embodiment, f( ) is the identity map. In another embodiment, f( ) is a morphological filter. The multiplication operation “*” is applied to change detection map C and image B on a pixel by pixel basis.

C′(t) can be losslessly compressed. The image B(t) is used for contextual information and can also be losslessly compressed. Multiple frames (for example, about 20 frames) of images B(t) can be time-averaged to generate a background image B′. In the embodiment in which 20 frames are averaged, a 20:1 compression ratio results. This background image B′ can further be spatially compressed by up to 60:1 compression ratio.

B′ and C′(t) can both be used for storage, transmission and reconstruction of the image at time t. For reconstruction at time t which lies within the 20 frame interval in which B′ is generated, B′ and C′(t) are both uncompressed and then combined according to the following formula:

Reconstructed Image=B′*(1−sign(B′*C′(t)))+B′*C′(t).

In the above formula, sign( ) is the signum function and the multiplication operation “*” is performed on a per pixel basis.

The foregoing compression procedure provides a significant reduction in data storage and transmission needs compared to traditional compression procedures.

In sample embodiments, e.g. in persistent surveillance, a single background image can be sufficient, and all subsequent image acquisition can be accomplished by applying the methods of the present invention. In other sample embodiments, e.g. in airborne applications with Time Delay Integration mode operation, the same geographic location can be sampled for a number of frames (e.g., 3 to 5 frames) and only 2 to 4 frames can be used for detection of moving objects.

Additional applications of the methods and devices disclosed herein include the following:

Automatic extraction of motion without tracking from markers for motion capture at lower capture rates

Optimized camera deshake/deblurring by automatic mover segmentation

Optimized sensing for high speed and high frequency events such as high velocity impact tests and mechanical assembly.

Task specific compression procedure, such as target specific compression procedures.

In various embodiments, the devices and methods described herein can be used to detect saccades, for example, of human subjects. Saccades detection, in turn, can be employed for various purposes, including, but not limited to, diagnosing certain conditions and disorders. Accordingly, in one embodiment, the present invention is a method of saccade detection in a subject. The example method comprises directing radiation reflected from at least one eye of the subject onto a detector, said detector having a frame rate; acquiring first and second complementary sub-images of a single frame, wherein the first and the second complementary sub-images are acquired at a sub-frame rate; and combining the first and the second complementary sub-images to detect the saccades in the subject.

In another embodiment, the present invention is a method of diagnosing a disorder in a subject in need thereof, the method comprising detecting saccades of the subject by directing radiation reflected from at least one eye of the subject onto a detector, said detector having a frame rate; acquiring first and second complementary sub-images of a single frame, wherein the first and the second complementary sub-images are acquired at a sub-frame rate; and combining the first and the second complementary sub-images to detect the saccades of the subject, wherein the condition is a traumatic brain injury, an attention deficit disorder, autism, dyslexia, multiple sclerosis and ocular palsy. Other applications of saccade detection include lie detection testing, security checking and identity testing.

### EXEMPLIFICATION

### Example 1

### Simulated Change Detection Map

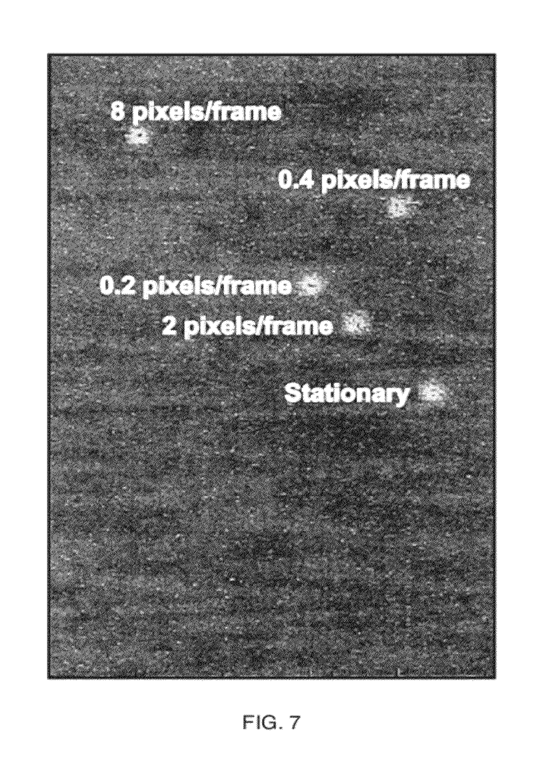

Moving targets were simulated as Gaussian shaped objects undergoing rigid body linear motion during a single image frame collection time. Each target is modeled as a Gaussian-shaped object embedded in background noise, which is defined as Gaussian distributed with 0 mean, standard deviation of 0.1. The results are shown in FIG. 7, which is a screen capture of an output of MATLAB simulation of several Gaussian-shaped targets undergoing a range of linear motions from multi-pixel to subpixel during a single image frame acquisition. The speeds of the targets, as indicated in pixels per frame, are also indicated in FIG. 7.

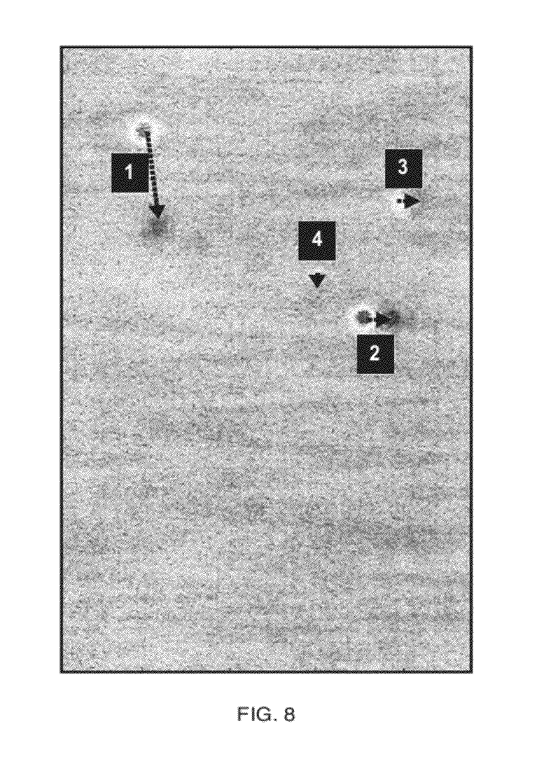

A change detection map of FIG. 8 was produced by the method of the present invention using the simulation shown in FIG. 7. The aperture modulation pattern was simulated as a dual rail modulation shown in FIG. 6B. Indicated are the derived heading vectors in image space.

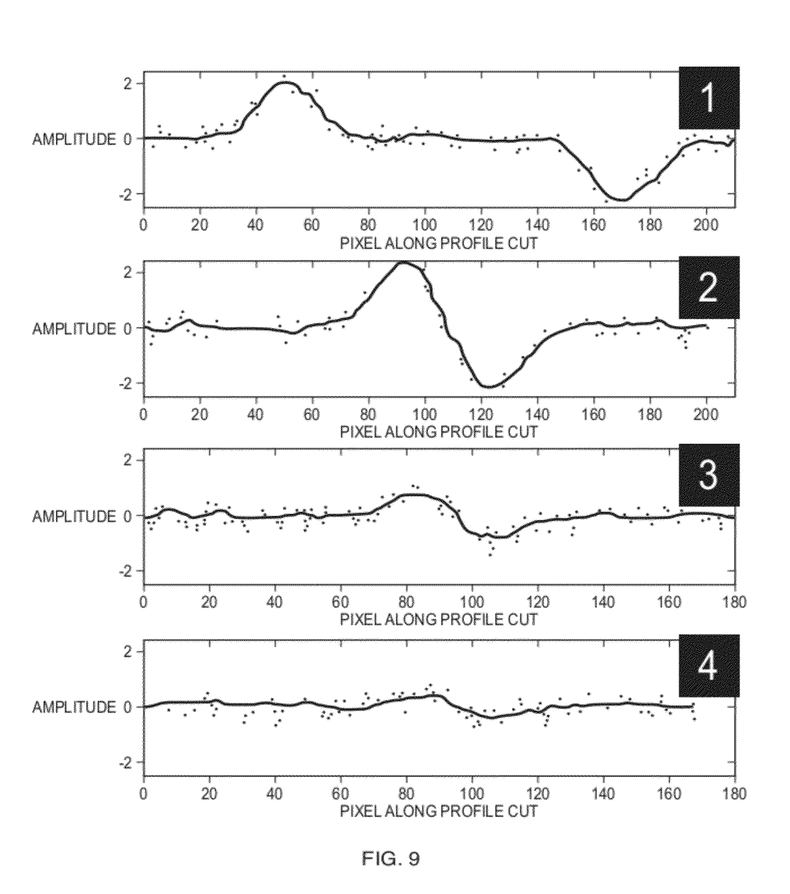

FIG. 9 includes multiple plots representing the associated profiles from the line cuts across each velocity detection shown in FIG. 8. Velocity in pixel coordinates is estimated by measuring peak to peak amplitude locations and dividing by the length of the modulation sequence. For subpixel motion, the existence of nonzero values in the resultant change detection map is used only to indicate the presence of subpixel movers without corresponding measure of velocity. This is the case for the first two targets (1)-(2). For the subpixel movers, the amplitudes of the profiles can be used as an indicator of subpixel motion with unreliable estimates of the velocity.

### Example 2

### Saccades Detection

Five human subject were subjected to verbal queries designed to elicit a variable saccade response. A high-speed camera (1000 frames per second) was used to detect saccades. An emulation of the operation of a device described herein was performed.

The emulation mimics the generation of two subimages on a single focal plane at 70 fps using the voltage profile for a polarizing element as shown in FIG. 11. The two subimages were subsequently subtracted to generate a change detection encoded image.

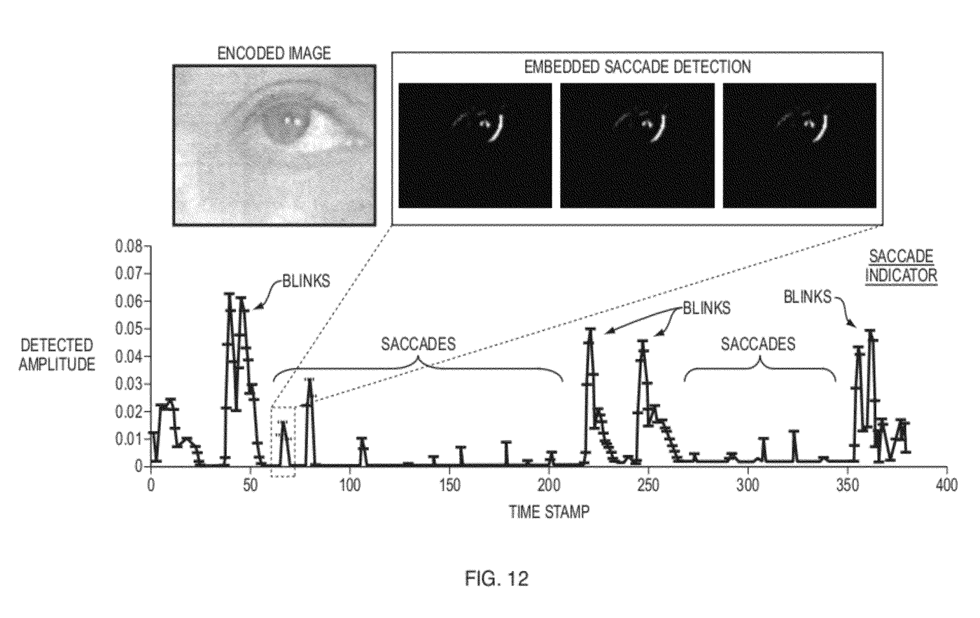

The resulting encoded image for a single frame is shown in FIG. 12. In the images shown in FIG. 12, motion presented itself as negative values because of the specific polarization modulation sequence selected. The images were then “thresholded” to generate a binary map indicating the presences of the saccades as shown in the sequences to the right. In FIG. 12, three continguous frames at 70 fps are shown. A simple saccade indicator is illustrated in the lower plot. Here, the normalized sum of all bright pixels per encoded image frame was used as metric for the presence and strength of a saccade. Blinks had a recognizable structure, whereas saccades appeared as impulsive responses with amplitude directly related to magnitude (angular extent of motion) of the saccade. From this simple metric, saccade latency, number, etc. can be derived for diagnostics.

Using this emulation, the proof of principle was achieved: the methods disclosed herein were capable of detecting saccades.

It should be understood that the methods disclosed herein may be performed, in part or in whole, by hardware, firmware or software. If performed by software, the software may be any language capable of performing the example embodiments disclosed herein. The software may be stored on a non-transient computer-readable medium, such as RAM, ROM, optical or magnetic disk, and be loaded and executed by a general or application-specific processor according to the example embodiments disclosed herein.

While this invention has been particularly shown and described with references to example embodiments thereof, it will be understood by those skilled in the art that various changes in form and details may be made therein without departing from the scope of the invention encompassed by the appended claims.

What is claimed is:

1. A method of producing a change detection map, the method comprising:
directing incident radiation onto a detector, said detector having a frame rate;
acquiring first and second complementary sub-images of a single frame, the first and the second complementary sub-images being acquired at a sub-frame rate; and
combining the first and the second complementary sub-images to yield the change detection map.

2. The method of claim 1, wherein directing incident radiation onto a detector includes directing the incident radiation through two or more modulated apertures.

3. The method of claim 2, wherein directing the incident radiation through two or more modulated apertures includes, during a single frame acquisition period of the detector:
opening the first aperture for combined duration of one or more time intervals, said time intervals forming a first set of time intervals, thereby forming a first beam;
opening the second aperture for combined duration of one or more time intervals, said time intervals forming a second set of time intervals, thereby forming a second beam; and
directing the first and, separately, the second beams onto the detector, thereby acquiring the first and the second complementary sub-images,
wherein the lengths of time intervals in each set of time intervals are modulated according to a dual-rail binary modulation pattern.

4. The method of claim 1, wherein acquiring the first and the second complementary sub-images includes, during a single frame acquisition period of the detector:
converting the incident radiation into first set of electric charges representing the first sub-image;
storing the first set of charges; and
converting the incident radiation into second set of electric charges representing the second sub-image.

5. The method of claim 4, wherein
acquiring the first and the second complementary sub-images includes directing the incident radiation at a detector array that includes a plurality of radiation exposure sites for converting the incident radiation into electric charges and a plurality of charge storage sites for storing electric charges; and
wherein storing the first set of electric charges includes transferring the first set of electric charges from the radiation exposure sites to the charge storage sites.

6. The method of claim 5, wherein:
converting the incident radiation into the first set of electric charges includes exposing the exposure sites to the incident radiation for combined duration of one or more time intervals, said time intervals forming a first set of time intervals;
converting the incident radiation into the second set of electric charges includes exposing the exposure sites to the incident radiation for combined duration of one or more time intervals, said time intervals forming a second set of time intervals,
wherein the lengths of time intervals in each set are modulated according to a dual-rail binary modulation pattern.

7. The method of claim 1, wherein acquiring the first and the second complementary sub-images includes directing the incident radiation through a modulated polarizing element.

8. The method of claim 7, wherein directing the incident radiation through the modulated polarizing element includes, during a single frame acquisition period of the detector:
imposing a first polarization onto the incident radiation for a combined duration of one or more first time intervals, said first time intervals forming a first set of time intervals, thereby forming a first beam having a first polarization;
imposing a second polarization onto the incident radiation for a combined duration of one or more second time intervals, said second time intervals forming a second set of time intervals, thereby forming a second beam having a second polarization; and
directing the first beam and, separately, the second beam onto the detector, thereby acquiring the first and the second complementary sub-images;
wherein the lengths of time intervals in each set of time intervals are modulated according to a dual-rail binary modulation pattern.

9. The method of claim 1, wherein combining the first and second complementary sub-images includes adding the first complementary sub-image from the second complementary sub-image.

10. The method of claim 1, wherein combining the first and second complementary sub-images includes subtracting the first complementary sub-image from the second complementary sub-image.

11. The method of claim 1, wherein combining the first and second complementary sub-images to yield the change detection map includes:
adding the first and second complementary sub-images to yield a complementary sub-image sum; and
integrating the complementary sub-image sum to yield the change detection map.

12. The method of claim 1, further including:
estimating motion of an object using the change detection map.

13. The method of claim 11, wherein the object moves by an amount corresponding to less than one resolvable spot of the detector.

14. An apparatus for acquiring an image, comprising:
a detector array configured to capture frames at a frame rate and to acquire first and second complementary sub-images, said detector array including a plurality of radiation exposure sites for converting the incident radiation into electric charges and a plurality of charge storage sites for storing electric charges, the detector array further configured to transfer, during acquisition of a single frame, the electric charges from the radiation exposure sites to the charge storage sites; and
a processor, operably coupled to the detector array, configured to combine the first and the second complementary sub-images to yield the change detection map.

15. The apparatus of claim 14, further including:
a combiner operably coupled to the detector array and configured to combine the first and second complementary sub-images to produce a change detection map.

16. The apparatus of claim 15, further including:
an integrator operably coupled to the combiner and configured to integrate an output of the combiner to form a change detection map.

17. The apparatus of claim 15, wherein the processor is operably coupled to the combiner and configured to estimate motion of an object using the change detection map.

18. The apparatus of claim 17, wherein the processor is further configured to estimate motion of an object wherein the object moves by an amount corresponding to less than one resolvable spot of the detector during acquisition of a single frame.

19. An apparatus comprising:
a first and a second detector array, each having a frame rate;
a first aperture configured to open for combined duration of one or more time intervals, said time intervals forming a first set of time intervals, thereby exposing the first detector to the incident radiation and capturing a first complementary sub-image;
a second aperture configured to open for combined duration of one or more time intervals, said time intervals forming a second set of time intervals, thereby exposing the second detector to the incident radiation and capturing a second complementary sub-image; and
a processor operably coupled to the first and second detector arrays and configured to combine the first and the second complementary sub-images to yield the change detection map.

20. The apparatus of claim 19, wherein the lengths of time intervals in each set of time intervals are modulated according to a dual-rail binary modulation pattern.

21. The apparatus of claim 19, wherein the first and second apertures are each independently actuated by shutters, each shutter independently selected from the groups consisting of a liquid crystal device, mechanical shutter, electro-optic device, and magneto-optic device.

22. The apparatus of claim 19, further including:
an integrator operably coupled to the combiner and configured to integrate an output of the combiner to form a change detection map.

23. The apparatus of claim 19, further including:
a processor operably coupled to the combiner and configured to estimate motion of an object using the change detection map.

24. The apparatus of claim 23, wherein the processor is further configured to estimate motion of an object wherein the object moves by an amount corresponding to less than one resolvable spot of the detector array during acquisition of a single frame.

25. An apparatus comprising:
a detector array having a frame rate;
a modulated polarizing element configured to (i) impose a first polarization onto the incident radiation for a combined duration of one or more time intervals, said time intervals forming a first set of time intervals, thereby forming a first beam having a first polarization and (ii) impose a second polarization onto the incident radiation for a combined duration of one or more time intervals, said time intervals forming a second set of time intervals, thereby forming a second beam having a second polarization;
an optical element configured to direct the first beam and, separately, the second beam onto the detector, thereby acquiring the first and the second complementary sub-images; and
a combiner, operably coupled to the detector array and configured to combine the first and the second complementary sub-images to yield the change detection map.

26. The apparatus of claim 25, wherein the lengths of time intervals in each set of time intervals are modulated according to a dual-rail binary modulation pattern.

27. The apparatus of claim 25, further including:
an integrator operably coupled to the combiner and configured to integrate an output of the combiner to form a change detection map.

28. The apparatus of claim 25, further including:
a processor operably coupled to the combiner and configured to estimate motion of an object using the change detection map.

29. The apparatus of claim 25, wherein the processor is further configured to estimate motion of an object wherein the object moves by an amount corresponding to less than one resolvable spot of the detector array during acquisition of a single frame.

30. An apparatus for producing a change detection map, the apparatus comprising:
means for directing incident radiation onto a detector, said detector having a frame rate;
means for acquiring first and second complementary sub-images of a single frame, the first and the second sub-images being acquired at a sub-frame rate; and
means for combining the first and second complementary sub-images to yield the change detection map.

31. An apparatus for producing a change detection map, comprising:
at least one detector array configured to acquire an image encoded in incident radiation, said detector array having a frame rate;
a modulator, configured to divide the image encoded in the incident radiation into the first and the second complementary sub-images during a single frame acquisition period of the detector; and
a combiner, operably coupled to the at least one detector array and configured to combine the first and the second complementary sub-images to yield the change detection map.

32. The apparatus of claim 31, further comprising a first and a second detector array, each having a frame rate, and wherein the modulator comprises:
a first aperture configured to open for a combined duration of one or more time intervals, said time intervals forming a first set of time intervals, thereby exposing the first detector to the incident radiation and capturing a first complementary sub-image; and
a second aperture configured to open for a combined duration of one or more time intervals, said time intervals forming a second set of time intervals, thereby exposing the second detector to the incident radiation and capturing a second complementary sub-image.

33. The apparatus of claim 31, wherein the modulator comprises a modulated polarizing element configured to (i) impose a first polarization onto the incident radiation for combined duration of one or more time intervals, said time intervals forming a first set of time intervals, thereby forming a first beam having a first polarization and (ii) impose a second polarization onto the incident radiation for combined duration of one or more time intervals, said time intervals forming a second set of time intervals, thereby forming a second beam having a second polarization,
the apparatus further including an optical element configured to direct the first beam and, separately, the second beam onto the at least one detector, thereby acquiring the first and the second complementary sub-images.

34. The apparatus of claim 31, wherein the modulator comprises a detector array configured to capture frames at a frame rate and to acquire first and second complementary sub-images, said detector array including a plurality of radiation exposure sites for converting the incident radiation into electric charges and a plurality of charge storage sites for storing electric charges, the detector array further configured to transfer, during acquisition of a single frame, the electric charges from the radiation exposure sites to the charge storage sites,
the apparatus further including a processor, operably coupled to the detector array, said processor configured to combine the first and the second complementary sub-images to yield the change detection map.

35. A method of diagnosing a disorder in a subject, the method comprising detecting saccades of the subject by:
directing radiation reflected from at least one eye of the subject onto a detector, said detector having a frame rate;
acquiring first and second complementary sub-images of a single frame, wherein the first and the second complementary sub-images are acquired at a sub-frame rate; and
combining the first and the second complementary sub-images to detect the saccades of the subject,
wherein the disorder is a traumatic brain injury, an attention deficit disorder, autism, dyslexia, multiple sclerosis or ocular palsy.

36. A method of detecting saccades in a subject, comprising:
directing radiation reflected from at least one eye of the subject onto a detector, said detector having a frame rate;
acquiring first and second complementary sub-images of a single frame, the first and the second complementary sub-images being acquired at a sub-frame rate; and
combining the first and the second complementary sub-images to detect the saccades in the subject.
